(19) United States
(12) Patent Application Publication
(10) Pub. No.: US 2013/0143319 A1
(43) Pub. Date: Jun. 6, 2013

(54) Use of Functional Nanoelectrodes for Intracellular Delivery of Chemical and Biomolecular Species

(71) Applicants: Min-Feng Yu, Champaign, IL (US); Kyungsuk Yum, Berkeley, CA (US)

(72) Inventors: Min-Feng Yu, Champaign, IL (US); Kyungsuk Yum, Berkeley, CA (US)

(73) Assignee: The Board of Trustees of the University of Illinois et al, Urbana, IL (US)

(21) Appl. No.: 13/634,978

(22) PCT Filed: Mar. 16, 2011
(86) PCT No.: PCT/US11/28701

§ 371 Date: Feb. 22, 2013

Related U.S. Application Data
(60) Provisional application No. 61/314,501, filed on Mar. 16, 2010.

(51) Int. Cl. A61N 1/32; C12N 5/09

(52) U.S. Cl. CPC A61N 1/325; C12N 5/0693
USPC 435/375; 604/501; 977/906

(57) ABSTRACT

The invention provides methods of controlled release of an agent into an intracellular environment of a biological cell using a needle nanoelectrode. The agent may be attached to an outer surface of the needle nanoelectrode through a linking molecule, wherein the attachment comprises an electroactive chemical bond. After penetrating a cellular membrane with the needle nanoelectrode to position at least a portion of the nanoelectrode in the intracellular environment, an electric potential may be applied to the needle nanoelectrode to break the electroactive chemical bond, thereby releasing the agent to the intracellular environment. The linking molecule may be a surface active organosulfur compound capable of forming a self-assembled monolayer on a metal surface of the nanoelectrode

### CROSS REFERENCE TO RELATED APPLICATIONS

This application claims the benefit of U.S. Provisional Application No. 61/314,501, filed Mar. 16, 2010, which is hereby incorporated by reference.

### STATEMENT REGARDING FEDERALLY SPONSORED RESEARCH OR DEVELOPMENT

This invention was made with government support under grant GM072744 awarded by National Institutes of Health and grants CBET-0933223 and CBET-073 awarded by the National Science Foundation. The government has certain rights in the invention.

### BACKGROUND

Yum et al. (Nano Letters, 2009, 9(5), 2193-2189) describe a method to deliver fluorescent quantum dots into living cells, using a membrane-penetrating solid nanoneedle. A NH₂-terminated self-assembled monolayer (SAM) was formed on a gold coated nanoneedle by chemisorption of thiols on gold. The NH₂ surface group was reacted with N-hydroxysulfosuccinimide esters of biotin, forming a stable amide bond. Streptavidin-coated quantum dots were attached onto the nanoneedle by the specific binding of streptavidin and biotin. The quantum dots conjugated on the nanoneedle were believed to be released by reductive cleavage of disulfide bonds.

US Patent Application Publication 2008/00678056 (Searson et al.). describes a method for controlled release of an agent such as a biomolecule or nanoparticles. The method involves linking the agent to molecules which are chemically bonded to the electrode surface and form a self-assembled monolayer. The agent may be electrochemically released from the electrode surface. The molecules chemically bonded on the surface of the electrode may be thiols on a gold electrode. The electrodes may be patterned on a substrate and the agent released into a liquid medium.

U.S. Pat. No. 7,597,950 (Stellaci et al.) describes methods for creating monolayer protected surfaces for surfaces having a local radius of curvature of less than or about equal to 1000 nanometers, including nanoparticle surfaces. Methods are described in which a first ligand and a second ligand are attached by self-assembly to the surface, wherein the first and second ligands are of different chain length and are selected and attached so as to form ordered domains having a characteristic size of less than or about equal to 10 nanometers, wherein the surface is the surface of a nanoparticle.

### BRIEF SUMMARY

In one aspect, the invention provides a method for controlled release of an agent into an intracellular environment of a biological cell. In an embodiment, the agent comprises a detectable tag such as a quantum dot (QD) or a magnetic nanoparticle. The ability to deliver a controlled number of monodispersed detectable tags into living cells with spatial and temporal precision can facilitate efficient targeting of the intended region of molecules and thus can allow spatially resolved molecular experiments inside cells.

In an embodiment, the methods rely on attachment of the agent to an electrode. In an embodiment, the delivery end of the electrode is nanoscale in diameter and “needle-like” in shape. The nanoscale portion of the electrode may be called a nanoelectrode. The electrode may have a “handle” portion which is of larger than nanoscale diameter. Nanoscale electrode tips have the potential to mechanically pass through cell membranes with minimal intrusiveness and locally deliver and release the target with sub-microscale precision.

In an aspect, insertion and/or removal of an electrode of the present invention into a biological cell or organelle thereof, does not permanently adversely affect the cell. In particular, interruption to the integrity of a membrane or other biological envelope is transient or sufficiently minor that the cell remains viable and does not suffer a significant increase in the likelihood or rate of cell death compared to a cell that has not undergone electrode insertion. Accordingly, the term “needle” as used herein reflects the aspect of the invention where the insertable portion of the electrode minimally effects biological tissue or cell, and is unlikely to permanently damage or other adversely affect the tissue or cell to which the electrode is introduced.

In an embodiment, the number of agents released is limited to minimize interference with cell functions or with cell dynamics experiments. One practical upper limit to the number of agents bound to a nanoelectrode is the available surface area to which the agent may be bound. In an aspect, the available surface area corresponds to the functionalized nanoelectrode surface area, which in turn is dependent on the diameter of the nanoelectrode and the functionalized length of the nanoelectrode. In different embodiments, the functionalized length of the nanoelectrode is less than or equal to 10 microns or less than or equal to 3 microns. In addition, the number of agents bound to the nanoelectrode is generally influenced by the surface density of agents in the functionalized region. When the agents are attached to the nanoelectrode by attachment to molecules forming a self-assembled monolayer, the agent surface density may be controlled by tailoring the self-assembled monolayer to affect agent surface density magnitude and/or spatial distribution thereof over the nanoelectrode surface.

In one aspect, agent surface density is relatively uniform over the functionalized surface area. In another aspect, agent surface area density spatially (e.g. axially) varies over the functionalized surface area. For example, there may be regions of high agent surface density and other regions of low agent surface density, wherein the ratio of high:low is about 100, about 10, or any range therebetween. As another example, there may be regions where different agents are present over the functionalized surface area. In one embodiment, variation in surface agent density magnitude or distribution is achieved by correspondingly varying underlying functionalization of the nanoelectrode surface. For example, in any of the processes provided herein, it may be desired to preferentially release agent to one or more specific positions in the cell while only using a single needle nanoelectrode. By spatially patterning the agent over the needle nanoelectrode surface, a single nanoelectrode may provide localized delivery to multiple specific intracellular sites simultaneously. In one embodiment, the end or tip portion of the nanoelectrode has a high agent surface density for preferentially releasing agent in a localized cell region that is adjunct to the tip. In an aspect, one or more select axial positions along the nanoelectrode may have high surface agent density, thereby providing a pattern of preferential intracellular agent release targeted to one or more organelles, or cytoplasm regions, of the biological cell. In this manner, agents may be simultaneously released to multiple targeted areas that are physically separated (e.g., nucleus, extracellular membrane, endoplasmic reticulum, mitochondria, golgi apparatus)

In an embodiment, the methods of the invention rely on electrochemically programmed release of the agent. Such electrochemically-based release methods allow rapid release of the agent, thereby minimizing unintended interference to cellular activities during the release process. In addition, electrochemically-based release methods allow quantitative control of the release of the agent through control of the magnitude and/or duration of the applied electrical potential

In different embodiments, the biological cell is in vitro, in vivo, or ex vivo. The biological cell may comprise an isolated cell and may remain viable during and after the agent release process. The intracellular environment may be the nucleus, other organelle, or the cytoplasm. The intracelluar environment may also be sub-nuclear.

In an aspect, the agent is a probe used to visualize or otherwise monitor the cell or a biological process therein.

In an aspect the agent is a therapeutic. Therapeutic is used broadly to refer to a composition that provides a benefit to the cell. In an aspect the therapeutic is a chemical compound or a drug useful in treating a disease state. In an aspect, the therapeutic is a biological entity such as a protein, polypeptide, antibody, polynucleotide such as DNA or RNA. In an aspect, the biological entity is packaged to facilitate delivery and/or incorporation into the biological cell. For example, the DNA may be packaged into a vector to facilitate controlled incorporation into the cellular genome. In an aspect, the agent is used for genetic engineering, wherein the cellular DNA is modified by introducing a piece of DNA that is not found in the native DNA, to express DNA that is not normally expressed, or to silence expression of DNA that is normally expressed.

In an aspect, the agent is a diagnostic, to identify or detect genetic mutations, diseased cells (e.g., cancerous cells or cells infected by a virus and/or bacteria), identify proteins such as proteins that are associated with bacterial or viral infection, or to identify any physical abnormality or defect in a cell.

### BRIEF DESCRIPTION OF THE DRAWINGS

FIGS. 1a and 1b. Electrochemically controlled delivery of QDs into a living cell. 1a) Schematic describing the delivery and release principle of cargo through a membrane penetrating nanoneedle: applying a potential larger than the critical potential V_{C} to the nanoneedle induces desorption of a SAM and thus release of QDs. 1b) Procedure for surface functionalization of a gold-coated nanoneedle and attachment of streptavidin-coated QDs.

FIGS. 2a and 2b. Optical microscope image of a typical gold-coated nanoneedle (2a) and scanning electron microscope image of a gold-coated nanoneedle (2b).

FIGS. 3a and 3b. Electrochemically controlled release of QDs from gold surfaces in Dulbecco's modified Eagle's medium (DMEM) as described in the Example. 3a) Fluorescence intensity versus applied potential on a gold surface functionalized with QDs via biotin-terminated thiols (biotin-terminated tri(ethylene glycol)hexadecanethiol) (top). The fluorescence of the gold surface was measured after applying a potential for 30 s sequentially. Applying a potential larger than the critical potential V_{C} (˜1.0 V) induced the desorption of the SAM and thus the attached QDs and their diffusion into the bulk solution, decreasing the fluorescence signal. Corresponding fluorescence images at the labeled electrical potentials of the gold surface in gray scale are shown at the bottom, showing gradual loss of the fluorescence. 3b) Fluorescence intensity versus time curve of a gold surface functionalized with QDs at a constant applied potential of 1.4V versus Ag/AgCl wire in DMEM. The fluorescence intensity decreased as the SAM and the attached QDs were desorbed and diffused into the bulk solution. Corresponding fluorescence images at the indicated time are shown at the bottom.

FIGS. 4a and 4b. 4a) Bright-field (top) and fluorescence (bottom) images of a nanoneedle before penetrating into a cell for the delivery experiment. The target cell is shown on the left side of the nanoneedle; the cell was unfocused because it was below the nanoneedle. The QDs attached on the nanoneedle are shown in white. Scale bar, 5 μm. 4b) Image of the nanoneedle during the delivery experiment. The whole process was monitored under the direct visualization of the optical microscope. The nanoneedle could be precisely located at the target release site in the three-dimensional cellular environment by focusing on the tip of the nanoneedle. The tip of the nanoneedle and the nuclear envelope is on the same focal plane. Scale bar, 10 μm. The unfocused dark shade on the right side of the nanoneedle in (4a) and (4b) is the macroscopic needle on which the nanoneedle was attached. The arrow indicates the tip of the nanoneedle; the dotted line guides the nanoneedle, gradually unfocused from its tip.

FIGS. 5a-5c. Delivery and tracking of single QDs inside a nucleus. 5a) Delivery of QDs) into the nucleus of a living HeLa cell: fluorescence image (upper, QDs are light gray and generally indicated by white arrows) and overlay of bright-field and fluorescence images (lower, QDs generally indicated by dark arrows) of the cell on a focal plane. The dotted line indicates the boundary of the nucleus. Scale bar, 10 μm. 5b) Typical time trace of the fluorescence intensity of a slow-moving QD (upper solid points), indicated by the rightmost arrow in (5a), showing the blinking pattern, plotted with the background signal of neighboring areas (black in 5a). 5c) mean square displacement (MSD) versus time data of QDs in the nucleus, showing three types of characteristic motions: free diffusion (dark gray, fit by upper curves), confined diffusion (fit by middle curve) and virtually stationary during the observation (black, fit by lower curve) (left). The solid lines are the fit of free and confined diffusion models.^{(9, 17)} The freely-diffusing QDs in the plot (gray) have D values of 0.3 and 2 μm²/s. Tracking of QDs in the nucleus, showing freely diffusing and confined QDs (right). Scale bar, 1 μm.

FIG. 6. Delivery of QDs into the cytoplasm of a living HeLa cell: fluorescence image (left, QDs indicated by bright spots) and overlay of bright-field and fluorescence images (right, arrows generally indicate position of QDs) of the cell on a focal plane. Scale bar, 10 μm.

### DETAILED DESCRIPTION

Typically, the electrode includes a nanoscale portion having a diameter or characteristic width less than or equal to 300 nm; the nanoscale portion of the electrode may be referred to as a nanoelectrode. In an embodiment, the nanoelectrode portion of the electrode is elongated along a longitudinal axis and “needle-like” in shape and is referred to as a “needle nanoelectrode”. The nanoelectrode may be solid, rather than having a hollow interior or may have a hollow interior which is sealed from contact with the intracellular environment. In an embodiment, the nanoelectrode cross-section perpendicular to the longitudinal axis is generally circular. The outer diameter of the nanoelectrode portion of the electrode may be constant or varying along the length of the nanoelectrode. For example, the nanoelectrode may be generally cylindrical in shape. A first portion of the nanoelectrode may also be generally cylindrical, with a second portion in the vicinity of the junction with the support being of larger diameter. The tip portion of such a nanoelectrode may be of smaller diameter. The nanoelectrode may also be smoothly or stepwise tapered. The diameter of a portion of the nanoelectrode may be from 50 to 300 nm, from 50 nm to 100 nm, or from 100 nm to 300 nm. In an embodiment, the delivery end of the nanoelectrode has a diameter between 10 nm and 300 nm. The aspect ratio of the nanoelectrode may be at least 10 times the diameter. The nanoelectrode is electrically conducting and is adapted for connection to a source of electrical potential. In an embodiment, a metallic portion of the nanoelectrode is exposed for functionalization with the SAM.

Furthermore, the nanoelectrode may be shaped to minimize or avoid permanent change or damage to a biological cell or tissue, or a constituent thereof. In this manner, the tip may be sharpened to a shape analogous to a needle to facilitate entry through the membrane, passage through the cytoplasm, and/or entry into an organelle without undue disruption. Elongation to a high aspect ratio of the needle nanoelectrode, where the diameter of the electrode portion that enters the cell is small, such as less than 1 μm, less than or equal to 300 nm, less than or equal to 100 nm, or from 1 nm and 10 nm (at the tip end that makes initial contact and entry with the extracellular membrane) and 100 nm to 500 nm (toward the other end that connects the nanoelectrode to the electrode handle) can further reduce unwanted disruption without compromising the integrity and fidelity of the nanoelectrode. The radius of curvature at the tip of the nanoelectrode may be from 20 to 100 nm or from 20 to 50 nm.

The nanoelectrode may comprise a nanotube or a nanotube coated with gold or another conductive film. Suitable films include gold, silver and platinum. The thickness of the conductive film may be from 5 to 20 nm. If the nanotube has a hollow interior, application of a conductive film to the nanotube may seal the end of the nanotube. The nanotube may be a boron nitride nanotube coated with a conductive metal film, such as a gold film. The length of such a nanoelectrode may be from 10 microns to 30 microns. Nanotube materials can exhibit extraordinary mechanical, electrical and/or chemical properties, which has stimulated substantial interest in developing applied technologies exploiting these properties. For example, nanotubes can have very high Young's modulus values. Multi-walled carbon nanotubes have been measured to have Young's modulus values between 0.1 and 1.33 TPa, with the Young's modulus being dependent upon the degree of order within the tube walls (Demczyk et al., 2002, Mater. Sci. and Engr. 1334, 173-178; Salvetat et al., 1999, Appl. Phys. A 69, 255-260). Multi-walled boron nitride nanotubes have been measured to have a Young's modulus of about 1.22 TPa (Chopra et al., 1998, Solid State Comm, 105(5), 297-300). Because of their high strength nanotubes have been suggested as reinforcements for composite materials.

As used herein, the term “nanotube” refers to a tube-shaped discrete fibril typically characterized by a substantially constant diameter of typically about 1 nm to about 100 nm, preferably about 2 nm to about 50 nm. In addition, the nanotube typically exhibits a length greater than about 10 times the diameter, preferably greater than about 100 times the diameter. The term “multi-wall” as used to describe nanotubes refers to nanotubes having a layered nested-cylinder structure. The layers are disposed substantially concentrically about the longitudinal axis of the fibril. A variety of multi-walled nanotube compositions are known to the art, including, but not limited to, carbon, boron nitride, carbon nitride, carbon boron nitride, and sulfides. In an embodiment, such a “nanotube” is used in this application purely from the consideration of its excellent mechanical property and its hollow nature is not exploited.

Boron nitride nanotubes comprise boron combined with nitrogen. In an embodiment, the nanotubes comprise essentially only boron and nitrogen. Boron nitride nanotubes may contain low levels of impurities or can be doped with other elements or molecules. Typically the concentration of dopants is less than 1%. Besides doping, nitrogen vacancies are also possible in boron nitride. Boron nitride nanotubes are inherently large band-gap semiconductors and thus almost insulators. Boron nitride nanotubes can be made by a variety of methods including arc discharge, laser heating, and oven heating. Boron nitride nanotubes have been reported to be a good dielectric material up to about 10V (Cumings, J. and Zettl, A., 2004, Solid State Communications. 129, 661-664).

The nanoelectrode can also comprise a conductive film coated nanowire or a conductive metallic nanowire. In an embodiment, the conductive film is a metallic film. The nanowire material may be selected for low toxicity in an intracellular environment. For example, the nanoelectrode may comprise a nanowire selected from the group consisting of platinum or platinum iridium alloys. The nanoelectrode may also comprise a nanowire of a first metal such as copper coated with a film of a second metal such as gold, silver, or platinum. The thickness of the conductive film may be from 5 to 20 nm. The length of such a nanoelectrode may be from 10 to 20 microns.

The electrode will typically further comprise a support portion connected to the nanoelectrode, at least a portion of the support portion having a larger diameter than the largest diameter of the nanoelectrode. The support portion may be at least partially electrically conductive and generally tapered in shape, with the nanoelectrode being located at the smaller diameter end of the support. For example, the nanoelectrode may be located at the tip or apex of a support. The support may be a sharpened tungsten tip. The nanoelectrode may be connected to the support portion by a conductive polymer-based adhesive material, by a metal film, by direct bonding between the materials of the nanoelectrode and the support portion, or combinations thereof.

For example, a nanoelectrode such as a boron nitride nanotube coated with a conductive metal film may be attached to a larger diameter “handle”, such as a sharpened tungsten tip. The attachment may be made with conductive glue such as a small droplet of silver paste or ultraviolet light curable conductive resin to provide both mechanical and electrical connection. Another metal film may be applied after the nanoelectrode is attached to the support, providing an additional connection between the nanoelectrode and the support.

In an embodiment, the nanoelectrode is a conductive metallic nanowire which is connected to a metallic region of the support through strong metallic bonds. Such a connection may be made by electrochemically depositing the nanowire on the metallic region of the support. The support as a whole may be metallic, may have a metallic portion or may be coated with a metallic film to provide the metallic region. U.S. Patent Application Publication 20090000364, hereby incorporated by reference, describes electrochemical deposition of nanowires, including metallic nanowires. Electrochemically deposited nanowires may have a generally cylindrical region uniform in diameter to within +/−15%, +/−10% or +/−5% and a base region in the vicinity of the junction between the nanowire and the support which is of larger diameter. The diameter of the tip region may be similar to that of the generally cylindrical region or may be sharpened to be of smaller diameter. For example, when the diameter of the generally cylindrical region is greater than 100 nm the tip region may be sharpened to have a radius of curvature from 20-50 nm. Metallic nanowires may be sharpened by focused ion beam milling or other methods known to the art. When the nanoelectrode is a metallic nanowire which is then coated with a layer of a second metal, the layer of the second metal may be applied after the nanoelectrode is attached to the support, providing an additional connection between the nanoelectrode and the support. Suitable supports for electrodeposition of metallic nanowires for electrode formation include, but are not limited to, metal wires which have been sharpened to a tip (e.g. through etching) or sharpened or unsharpened metal wires whose sides are coated by glass or another electrically insulating material. Such glass encapsulated metal wires can be made by “pulling” a glass micropipette with a metal wire inside, thereby reducing the diameter of the pipette and the metal wire inside. The pipette diameter may be reduced to a couple of micrometers or less. Electrical insulation of part of the support can significantly reduce the amount of exposed conductive surface potentially in contact with the extracellular media. The longitudinal axis of the nanowire may be aligned with the longitudinal axis of the support.

In different embodiments, the agent species may be a chemical species or a biomolecular species. In different embodiments, the agent species may be immobilized small molecules (e.g. drugs, chemical compounds), biopolymers, biologics (e.g. peptides, polypeptides, proteins, DNA, RNA, antibodies), protein assemblies (e.g. viruses), vectors, plasmids, or nanoparticles. “Biological molecule” is used broadly to refer to an agent that has an at least partially biologically-based composition (e.g., nucleotides, peptides, polynucleotides, polypeptides, genes, gene fragments, or compositions that are made by biological cells). Any one or more of these agent species may be packaged to facilitate delivery to regions of interest in the cell, including by normal cellular processes. In an embodiment, the agent is functionalized for attachment to the nanoelectrode. In an embodiment, the agent may be functionalized with a biotin-binding agent. In an embodiment, the agent may be functionalized to bind a structure of interest within the cell. For example, the agent may be functionalized with an antibody.

In an embodiment, the agent comprises a detectable tag. For example, the agent species may be an antibody that is itself labeled, such as with a fluorescence marker or radiolabel, a quantum dot, or a nanoparticle. In an embodiment, the agent may comprise particles which are fluorescent, magnetic, radioactive, electrically conducting, or absorptive/colored. In an embodiment, the particles are nanoparticles. As used herein, nanoparticles have an average size greater than or equal to 1 nm and less than 1000 nm. In an embodiment, the average size of the nanoparticles is 10-20 nm. When the agents comprise a detectable tag, the methods of the invention may include detecting the detectable tag to monitor the distribution of the agent in an intracellular environment.

In an embodiment, the particles are fluorescent particles. Fluorescent particles known to the art include fluorescently labeled microspheres and nanospheres. These particles include surface labeled spheres, spheres labeled throughout, and spheres possessing at least one internal fluorescent spherical zone (as described in U.S. Pat. No. 5,786,219 to Zhang et al.) Other fluorescent particles known to the art include quantum dots (QDots or QDs). These include naturally fluorescent nanoparticles that have optical properties that are tunable with their size. Commercially available quantum dots include nanometer-scale particles comprising a core, shell, and coating. The core may be made up of a few hundred to a few thousand atoms of a semiconductor material (often cadmium mixed with selenium or tellurium). A semiconductor shell (which may be zinc sulfide) surrounds and stabilizes the core. An amphiphilic polymer coating may encase this core and shell, providing a water-soluble surface that can be differentially modified. Commercially available quantum dots include QDots®, available from Invitrogen, which have reported peak emission wavelengths at 565 nm, 605 nm, 625 nm, 655 nm, 705 nm, and 800 nm. The quantum dots may be functionalized. For example, the QDs may be functionalized with a biotin binding protein such as streptavidin, a biomolecule such as an antibody, a target molecule complex or combinations thereof.

In an embodiment, the agent is attached to the outer surface of the nanoelectrode through a linking molecule, wherein the attachment comprises an electroactive bond. In an embodiment, the linking molecule chemisorbs to the outer surface of the nanoelectrode, forming a chemical bond. In an embodiment, the linking molecule comprises a surface active organosulfur compound capable of forming a self-assembled monolayer on a transition metal surface of the nanoelectrode. As used herein, a self assembled monolayer (SAM) is an ordered molecular assembly formed by the chemisorption of a surface-active agent on a solid surface. Surface-active organosulfur compounds known to form monolayers on gold surfaces include, but are not limited to alkanethiols, dialkyl disulfides, dialkyl sulfides, alkyl xanthates, and dialkylthiocarbamates (Ulman, 1996, Che. Rev. 96, 1533-1554). In an embodiment, the linking molecule comprises a sulfur binding group, a spacer chain, and a functional head group. In an embodiment, the sulfur binding group is a thiol, the functional end group is biotin, and the spacer chain comprises methylene groups (CH₂)ₙ or methylene groups and ethylene glycol groups (OCH₂CH₂)ₘ. The biotin end group may be modified for attachment to the rest of the linking molecule. Other groups may be present in the spacer chain. In an embodiment, the spacer group includes a (CH₂)ₙ segment where n is from 5 to 20. The spacer group may include a (CH₂)ₙ segment where n is from 5 to 20 and a (OCH₂CH₂)ₘ segment where m is from 3 to 6. The (CH₂) segment may be attached to the thiol binding group and the (OCH₂CH₂)ₘ segment attached to or near the functional end group. In an embodiment, the self-assembled monolayer is formed on a surface of the nanoelectrode, from a mixture of alkanethiol-type molecules functionalized with a binding moiety and alkanethiol-type molecules which are not functionalized with a binding moiety. As used herein, alkanethiol-type molecules include a thiol binding group and methylene groups in the spacer chain. The molar percentage of the binding moiety functionalized alkanethiol molecules, relative to the total amount of alkanethiol molecules, may be from 5% to less than 100 or from 5% to 30% depending on the size and amount of the agents to be attached. The molar percentage of the binding moiety functionalized alkanethiol molecules can be adjusted to obtain reasonable efficiency of molecular recognition between the binding moiety and the functionalized agent and also to obtain the desired loading of the agent. The spacer chain of the alkane thiol molecules may be the same length or similar lengths. For example, the spacer chain of the alkane thiol molecules may contain (CH₂)ₙ segments of the same length (value of n). In an embodiment, the spacer chain of the binding moiety functionalized alkane thiol molecules is longer than that of the alkanethiol molecules which are not functionalized with a binding moiety. In an embodiment, the end group of the alkanethiol molecule without the binding moiety is hydrophilic but has no specific binding affinity with streptavidin. Hydrophilic end groups on the alkane-thiol molecules known to the art include, but are not limited to hydroxy, carboxylate, and amine. As is known to the art, SAMs composed of a mixture of chemically different surface-active agents can be produced in either one step, by absorption from a solution of different molecules, or in two steps, by placing a preformed monolayer into a solution of a different surface-active agent.

Typically, self-assembled monolayers are prepared by immersing a substrate in a dilute solution of the surface active agent. The initial monolayer formed may be disordered, with ordering and packing density improving with increased immersion time. In different embodiments, the concentration of surface active agent in the solution is 0.5 mM or from 0.25 nM to 0.75 nM. In an embodiment, the solvent is an alcohol such as ethanol. In different embodiments, the immersion time is 12 hours or 6 to 24 hours. Typically, only a portion of the nanoelectrode is immersed in the solution during formation of the self-assembled monolayer. In different embodiments, only the last 3 microns or 10 microns of the tip region of the nanoelectrode is used as the attachment region, or the attachment contact area is less than or equal to 3 square microns.

For small diameter nanoelectrodes, the surface tension present at the solution surface can exert an appreciable force on the nanoelectrode during the nanoelectrode insertion and immersion process. In an embodiment, the nanoelectrode is pre-wetted with water or a solvent and is inserted at 45° (or within +/−15 degrees of 45°) to the surface of the solution to thereby reduce breakage of the nanoelectrode. In order to provide good control over the immersion process, a micromanipulator operably connected to the nanoelectrode may be used to dip the nanoelectrode in the solution containing the surface active agents.

The packing density of thiol molecules may be influenced by the substrate surface and the composition of the thiol molecule. A value of 7.7×10⁻¹⁰ mol/cm² has been reported for radiolabeled alkanethiol monolayers on gold (Schlenoff et al., 1995, J. Am. Chem. Soc, 117, 12528-12536). In one embodiment, the concentration of agent moieties in the functionalized region is 1.0×10⁻¹⁰ mol to 10.0×10⁻¹⁰ mol per 1 cm². In this manner, the self-assembled monolayer composition or concentration on the nanoelectrode surface is optionally spatially varying, such as by varying immersion time or packing density of thiol molecules by different processing of specific nanoelectrode surfaces. Spatially-varying the monolayer over the surface of the nanoelectrode thereby influences agent density and distribution over the nanoelectrode surface to facilitate simultaneous release of agents in geographically distinct locations within the cell.

In one embodiment, the functionalized area and concentration of agent moieties are selected together to produce a number of agent moieties less than or equal to 1 femtomole. In other embodiments, the number of agent moieties is from 1×10⁻¹⁹ moles to 1×10⁻¹⁶ moles or from 1×10⁻¹⁹ moles to 1×10⁻¹⁷ moles.

The self-assembled monolayer may include biotin-functionalized surface-active molecules. In this embodiment, the agent may be functionalized with a biotin-binding agent and bound to surface-active molecules in the SAM through binding between biotin and the biotin binding agent. In an embodiment, the biotin-binding protein comprises avidin, streptavidin, or NeutrAvidin. An agent functionalized with a biotin-binding agent may be attached to biotin-labeled molecules in the SAM by contacting the SAM with a solution including the functionalized agent. The solution may also include components to limit non-specific adsorption.

The attachment of the agent to the nanoelectrode comprises an electroactive chemical bond. To release the agent from the nanoelectrode, sufficient electrical potential is applied to the nanoelectrode to break the electroactive chemical bond. In an embodiment, the electroactive chemical bond is a bond between the metal surface of the nanoelectrode and a sulfur atom of the surface active agent forming the monolayer. The critical potential at which desorption of the surface active agents begins is typically dependent on the nature of the surface active agent as well as on pH. Typically the absolute value of the maximum applied potential will be greater than this critical potential. The applied potential may be positive, negative, or a combination of positive or negative pulses. In an embodiment, the applied potential is negative with respect to a reference electrode. In an embodiment, the potential required is sufficiently low that it does not permanently or adversely affect the cell. In an embodiment, the absolute value of the applied potential is in the range from 0.1 to 1.5 V, from 0.5 to 1.5V, and from 1.0 to 1.5V. In an embodiment, the value of the potential is selected so that evolution of hydrogen gas does not occur.

A two-electrode configuration may be used, the potential being applied between the nanoelectrode and a second electrode acting as a counter/reference electrode immersed in the cell medium. The counter/reference electrode may be Ag/AgCl or Pt wire. In different embodiments, the surface-active agents with attached agent species are completely desorbed in 90 seconds or less or 60 seconds or less, or between about 30 seconds and 90 seconds upon applying an electrical potential. If the loading of the electrode is sufficiently high, complete desorption of the agent species may not be required to obtain the desired amount or concentration of agent species within the cell. For example, desirable amounts of agent species may be released at times from 5 to 60 seconds, from 5 to 30 seconds, from 1 to 60 seconds, from 1 to 30 seconds or from 1 to 15 seconds. In an embodiment, at least some of the agents are delivered in single form, rather than in clusters.

As used herein, “viable” refers to a cell that does not experience a significant increase in cell death, such as by apoptosis or necrosis. In particular, a cell undergoing a process disclosed herein is said to remain viable if there is not a significant change in cell death compared to an equivalent cell that has not undergone the process.

In one aspect, the invention provides a method of controlled release of an agent into an intracellular environment of a biological cell, said method comprising the steps of:

a. providing an electrode comprising a nanoelectrode, at least a portion of the nanoelectrode surface being metallic;

b. attaching the agent to an metallic surface of the nanoelectrode through a linking molecule, wherein the attachment comprises an electroactive chemical bond;

c. penetrating a cellular membrane with the nanoelectrode to position at least a portion of the nanoelectrode in the intracellular environment; and

d. applying an electric potential to the nanoelectrode to break the electroactive chemical bond, thereby controllably releasing the agent to the intracellular environment.

In the aspect of the invention described in the previous paragraph, the electrode will typically further comprise a support portion connected to the nanoelectrode, at least a portion of the support portion having a larger diameter than the largest diameter of the nanoelectrode. The support portion may be at least partially electrically conductive and generally tapered in shape, with the nanoelectrode being located at the smaller diameter end of the support. The nanoelectrode may be connected to the support portion by a conductive polymer-based adhesive material, by a metal film, by direct bonding between the materials of the nanoelectrode and the support portion, or combinations thereof. At least a portion of the nanoelectrode may have an average diameter from 50 to 300 nm, 50 to 100 nm or 100 to 300 nm. This portion of the electrode may be generally cylindrical or tapered. The nanoelectrode may be from 10 to 30 microns or 10 to 20 microns in length, with the length being adjusted depending on the stiffness of the nanoelectrode. The nanoelectrode may be solid (rather than hollow) or may be hollow but sealed at its tip so that hollow portion of the nanoelectrode is not in communication with the intracellular environment. The nanoelectrode may comprise a nanotube or a metallic nanowire; the nanotube or nanowire may be coated with a metallic coating. Metallic nanowires formed through electrochemical deposition will typically be connected to the support by bonding of the metallic nanowire to a metallic portion of the support and may have a generally cylindrical region uniform in diameter to within +/−15%, +/−10% or +/−5% and a base region in the vicinity of the junction between the nanowire and the support which is of larger diameter. The diameter of the tip region may be similar to that of the generally cylindrical region or may be sharpened to be of smaller diameter. For example, when the diameter of the generally cylindrical region is greater than 100 nm the tip region may be sharpened to have a radius of curvature from 20-50 nm.

In this same aspect of the invention, the linking molecule may comprise a surface active compound capable of forming a self assembled monolayer (SAM) on a metal surface of the nanoeletrode, the surface active compound being chemisorbed to the metal surface by an electroactive chemical bond. The linking molecule may comprise a sulfur binding group, a spacer chain, and a functional head group. In an embodiment, the sulfur binding group is a thiol, the functional end group is biotin, and the spacer chain comprises methylene groups (CH₂)ₙ or methylene groups and ethylene glycol groups (OCH₂CH₂)ₘ. The biotin end group may be modified for attachment to the rest of the linking molecule. The spacer group may include a (CH₂)ₙ segment where n is from 5 to 20 or a (CH₂)ₙ segment where n is from 5 to 20 and a (OCH₂CH₂)ₘ segment where m is from 3 to 6. The linking molecule may be attached by forming a self-assembled monolayer on the metallic surface of the nanoelectrode from a mixture including alkanethiol-type molecules functionalized with a biotin moiety and then attaching a biotin-binding protein functionalized agent moiety to at least a portion of the biotin-functionalized alkanethiol-type molecules. The mixture may include alkanethiol-type molecules functionalized with a binding moiety, such as a biotin moiety, and alkanethiol-type molecules which are not functionalized with a binding moiety. The molar percentage of the binding moiety functionalized alkanethiol molecules, relative to the total amount of alkanethiol molecules, may be from 5% to less than 100% or from 5% to 30% depending on the size and amount of the agent moieties to be attached. The functionalized length of the nanoelectrode may be less than or equal to 3 microns. The portion of the nanoelectrode inserted into the cell has agents attached to its surface.

In this same aspect of the invention, the absolute value of the applied potential is greater than a critical voltage and may be in the range from 0.1 to 1.5 V, from 0.5 to 1.5V, or from 1.0 to 1.5V with respect to an Ag/AgCl reference electrode. In an embodiment, the applied potential may be negative with respect to the reference electrode. In an embodiment, the potential required is sufficiently low that it does not permanently or adversely affect the cell. In an embodiment, the value of the potential is selected so that evolution of hydrogen gas does not occur. The voltage may be applied for 30 seconds to 90 seconds, from 5 to 60 seconds, from 5 to 30 seconds, from 1 to 60 seconds, from 1 to 30 seconds or from 1 to 15 seconds.

In this same aspect of the invention, the biological cell may be in vitro, in vivo, or ex vivo. The biological cell may comprise an isolated cell and may remain viable during and after the agent release process. The intracellular environment may be the nucleus, other organelle, or the cytoplasm. The intracelluar environment may also be sub-nuclear.

In this same aspect of the invention, the agent may be a probe, a therapeutic or a diagnostic. The agent may comprise particles which are fluorescent, magnetic, radioactive, electrically conducting, or absorptive/colored. The particles may be nanoparticles, with suitable nanoparticles including quantum dots and magnetic particles.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination. One of ordinary skill in the art will appreciate that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In general the terms and phrases used herein have their art-recognized meaning, which can be found by reference to standard texts, journal references and contexts known to those skilled in the art. Any preceding definitions are provided to clarify their specific use in the context of the invention.

### Example

### Introduction of Agents to the Nucleus

Site-specific delivery of biological probes into a living cell has been a major challenge in experimental cell biology. We report the use of a membrane-penetrating nanoneedle (doubly serving as an electrode) for carrying a cargo into the nucleus and the rapid release of the cargo via an electrochemical reaction activated by an electrical potential. We demonstrate the delivery of single quantum dots into the nucleus and their tracking in the nucleoplasm with minimal fluorescent cross-interference. This ability to directly deliver biological probes into the nucleus with high spatial and temporal precision offers new strategies to study biology in a living cell.

It is becoming increasingly clear that the nuclear architecture plays an essential role in the genome maintenance and functions.⁽¹⁾ However, the study of nuclear structures, dynamics, and functions presents a major challenge to biological scientists. The key challenge is the imaging of nuclear components using conventional probes, such as fluorescent dyes or proteins. Such probes provide weak signal and tend to rapidly photobleach (typically <10 s), which limits both the temporal and spatial imaging resolution. An alternative is to use the bright probes such as quantum dots (QDs). However, to get such relatively large-sized probes into the nucleus requires overcoming the physical barriers of both the plasma membrane and the nuclear envelope without imposing unintended side effects.⁽²⁾ Herein we present a nanoneedle-based electrochemically-controlled delivery method that can potentially circumvent these limitations.

With their bright fluorescence, photostability, and nanoscale size, QDs have emerged as an alternative probe that complements fluorescent dyes and proteins.⁽³⁻⁵⁾ One of the most promising applications of QDs is molecular imaging in living cells.^{(3, 4, 6-11)} However, realization of the full potential of QDs for molecular imaging faces several problems, including the relatively large size of QD-biomolecule conjugates and QD-target molecule complexes,^{(6, 10, 11)} the lack of strategies for targeting intracellular biomolecules,⁽⁴⁾ the instability of the antibody-mediated targeting,^{(6, 11)} QD multivalency,⁽¹⁰⁾ and the intracellular delivery of QDs.^{(2)-5, 9, 12, 13)}

In particular, the intracellular delivery of singly-dispersed QDs into the cytoplasm or organelles, such as the nucleus, remains a major challenge for live cell studies.^{(2-5, 9, 12, 13)} Because of this delivery problem, except for a few studies,⁽⁸⁾ the use of QDs for molecular imaging in living cells has been mostly limited to visualizing plasma membrane proteins^{(6, 7, 9-11)} and biological processes related to endocytosis.^{(13, 14)} The targeted delivery of QDs into the nucleus of living cells is even more challenging (as further discussed below), and the use of QDs within the nucleus is scarce.⁽²⁾

Existing cellular delivery methods often introduce damage to the targeted cells, are spatially, temporally and quantitatively nonspecific, or rely on the endocytic pathway to transport biological probes,^{(5, 12, 15)} which significantly limits their application. For example, although various methods have been explored to deliver QDs into the cytoplasm of living cells, including peptide-, protein-, or polymer-mediated delivery and electroporation, QDs introduced by these methods either form aggregates or are sequestrated in vesicles via the endocytic pathways, losing the size advantage of QDs and precluding their more advanced use in the cytoplasm (e.g., labeling individual biomolecules or organelles and probing the intracellular environment).^{(5, 12, 13)} Although useful for whole-cell labeling, these methods require complex strategies to let QDs escape the endocytic pathways for studying subcellular or molecular phenomena.^{(3, 4, 12)}

The nuclear delivery of QDs presents additional complexities: if the QDs are to be first introduced into the cytoplasm in a singly-dispersed form, they need to escape the endocytic pathway, and then require a mechanism to facilitate their transport to the nucleus.⁽²⁾ Several research groups have demonstrated the targeted nuclear delivery of QDs by using nuclear localization signal (NLS) peptides.^{(2, 12, 16)} For example, the QDs conjugated with NLS peptides were first introduced into the cytoplasm via microinjection or electroporation, and then transported by the protein nuclear trafficking process to the nucleus.^{(2, 12)} (However, this strategy can only deliver small QDs (<˜20 nm in overall diameter, depending on the cell type) limited by the nuclear pore complex (20-50 nm in diameter); thus, its efficiency strongly depends on the size of NLS-QD complexes and the cell type and cycle.⁽²⁾ For instance, a study reported that in some HeLa cells all NLS-QDs (˜10-15 nm in diameter) could enter the nucleus, while in some other HeLa cells in the same delivery experiment all NLS-QDs were stuck in the perinuclear region. Commercial QDs conjugated with NLS often have diameters larger than ˜25 nm (including the peptide) and can not transverse the nuclear envelope.⁽²⁾ Such a size requirement makes it difficult to incorporate additional functionalities onto QDs by conjugating biomolecules, such as antibodies, or to directly use the commercially-available QDs (e.g., streptavidin-conjugated QDs used in this study) for broad applications. Furthermore, because of the lack of spatial, temporal, and quantitative control, the NSL-mediated delivery has only been demonstrated to track the nucleus with QDs, but not to observe subnuclear events.^{(2, 12)}

In this regard, direct delivery methods, such as microinjection and nanoneedle-based delivery, have shown better performance: they can deliver homogeneously or sparsely dispersed QDs directly into the cytoplasm and the nucleus with no need for endosomal escape.^{(5, 12, 17)} However, the relatively large size and tapered shape of the injection pipette makes microinjection liable to cell damage, especially for the nuclear delivery into small cells. The recently developed nanoneedle-based intracellular delivery method⁽¹⁷⁻¹⁹⁾ has the potential to achieve the best outcome for the targeted nuclear delivery as it is capable of mechanically passing through both the cellular and nuclear membranes with minimal intrusiveness, and locally delivering and releasing the attached probes with sub-microscale precision. However, the previously reported nanoneedle-based method for the intracellular delivery relies on the endogenous regulatory mechanism of cells to break the disulfide bonds in the linker molecules between the cargo and the nanoneedle.^{(17, 18)} This release process is thus not under any external control and takes over 15 minutes as required.^{(17, 18)} This relatively long incubation time needed for the passive release may increase the unintended interference to the cellular activities as the nanoneedle is maintained inside the cell for the whole duration and lose the data collection capacity during this period. The lack of any control on the release process also limits the scope of the cellular studies that can otherwise be carried out with the assistance of this delivery technology.

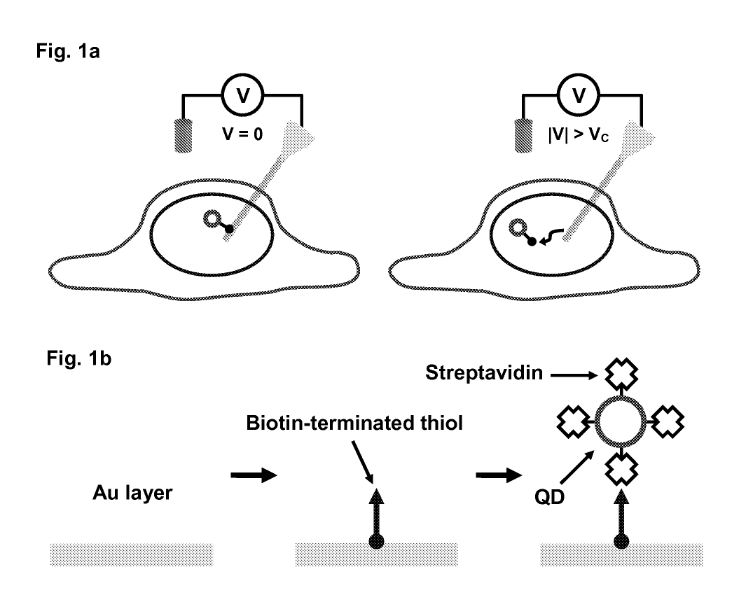

Here we present a controlled delivery and release method that uses a cargo-carrying nanoneedle (doubly serving as a nanoscale electrode) to penetrate into the nucleus of a living cell. To rapidly release the attached cargo inside the nucleus, we exploited an electrochemical means to directly break the incorporated electrochemically-active bonds by applying a small external electrical potential through the nanoneedle (FIG. 1a). The release of the cargo is thus almost instantaneous and is externally controlled by adjusting the magnitude and duration of the applied potential.

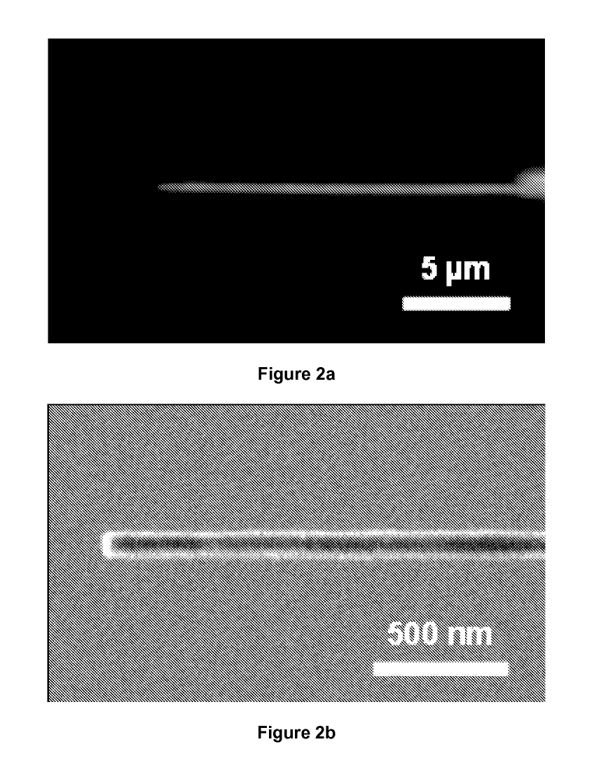

We used an individual boron nitride nanotube (˜50 nm in diameter) coated with a thin layer of gold (10-20 nm in thickness) as a nanoneedle (and an electrode), which was then attached onto a conductive sharpened wire for easy handling (FIG. 2).^{(17, 20)} The nanotube is chemically synthesized and has a uniform diameter, high mechanical strength and resilience, ideal as a membrane-penetrating nanoneedle as reported previously.^{(17, 20)} We attached a limited amount of cargo (streptavidin-conjugated QDs) by conjugating it on a self-assembly monolayer (SAM) formed on the approximately 2 micron long end segment of this gold-coated nanoneedle (FIG. 1b). The delivery of such tiny quantities of cargo limited by the nanoscale size of the nanoneedle minimizes their interference with cell dynamics experiments or with cell physiology. For example, excess QDs delivered into cells can present background interferences from out-of-focus QDs, decreasing the signal-to-noise ratio and thus making it difficult to track single QDs, besides interfering with cellular functions.⁽¹⁴⁾ More problematically, QDs are difficult to wash away once they are introduced into cells.⁽³⁾

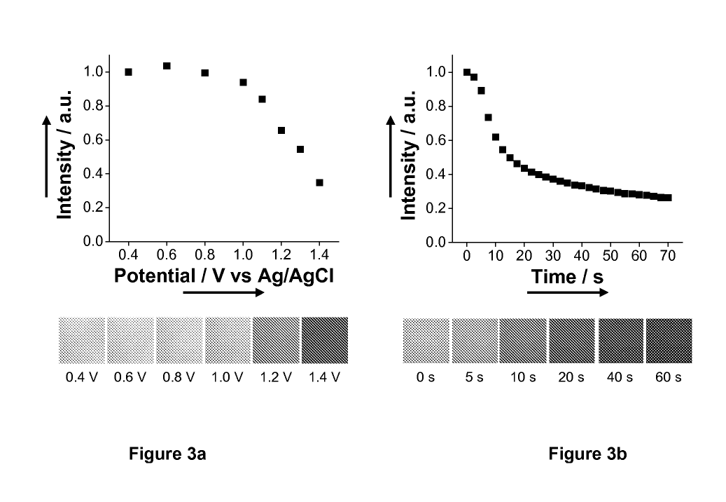

For the controlled release of cargo, we exploited the electrochemical desorption of the SAM from the gold surface (FIG. 1a and FIGS. 3a-3b).⁽²¹⁻²³⁾ A two-electrode configuration was used with the nanoneedle as a working electrode and a Ag/AgCl or Pt wire immersed in the cell medium as a counter/reference electrode.⁽²⁰⁾ To determine the critical potential (versus the reference electrode in the medium) and the kinetics of the QD release from the gold surface, we measured the fluorescent intensity of QD functionalized gold surfaces in the medium as a function of the increasing electrical potential applied on the gold surface, as shown in FIG. 3a; and as a function of time when applying a constant electrical potential sufficient for the release of the QDs (1.4 V versus Ag/AgCl wire in the medium), as shown in FIG. 3b. These control experiments show that the onset of the QD release occurs at an electrical potential of ˜1.0 V and the release can be completed within ˜60 s. Because of the different ionic environment in the cytoplasm and the nucleus (i.e., membrane potentials), the actual critical potential and the kinetics of the QD release inside the nucleus would be somewhat different from those obtained from these control experiments. However, because of the small membrane potential (˜0.1 V) compared to the applied potential of ˜1.0 V, its effect should not be significant for the release of QDs within the nucleus, as shown in our experiment later.

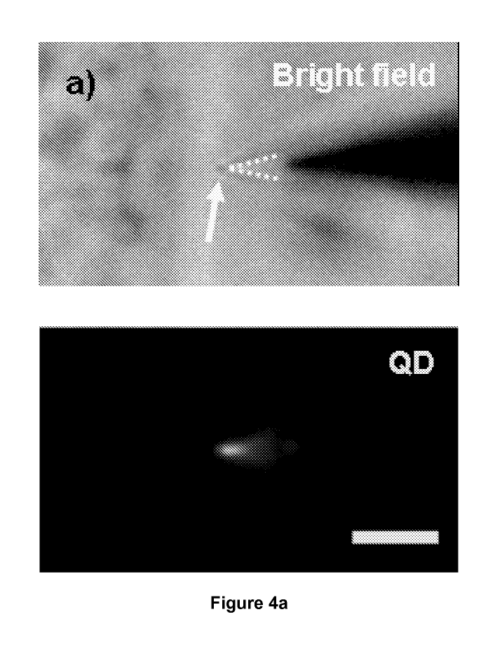

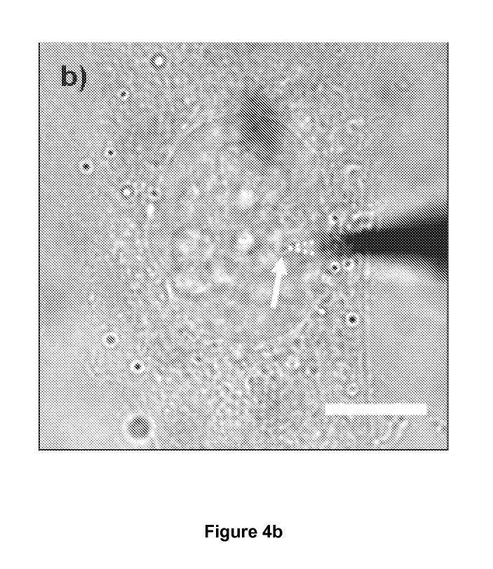

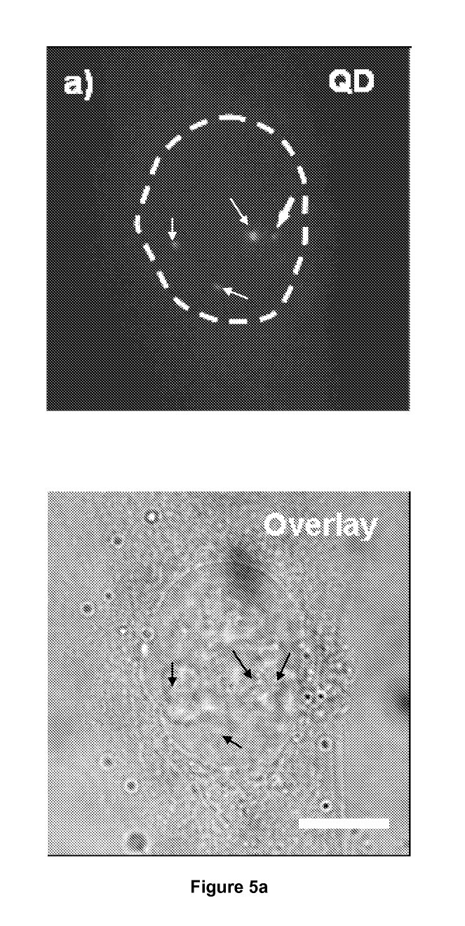

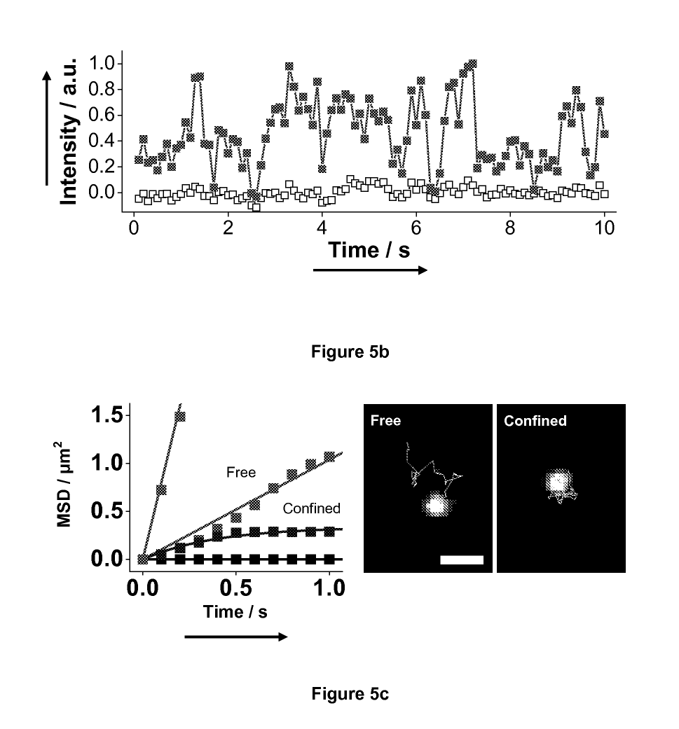

We demonstrate this method by delivering QDs into the nucleus of living HeLa cells (FIGS. 4a-4b and FIG. 5a). We used streptavidin-conjugated QDs with diameter of ˜15-20 nm (Qdot 655 streptavidin conjugates, Invitrogen). Notably, a previous study indicated that such commercial QDs conjugated with NLS peptides (>˜25 nm in overall diameter including the NLS peptides) did not enter the nucleus by the NLS-mediated delivery.⁽²⁾ We did the delivery experiment using a micromanipulator (InjectMan NI 2, Eppendorf) integrated in an inverted fluorescence microscope (Leica) as described previously.⁽¹⁷⁾ Once the nanoneedle pierced through the plasma membrane and the nuclear envelope and reached the target area, a small electrical potential was applied to the nanoneedle (typically, <1.4 V versus Ag/AgCl wire for 30-90 s) to release the SAM and thus the QDs; either a positive (oxidative) or negative (reductive) potential can be applied to release the QDs.⁽²¹⁻²³⁾ Once the desired release was completed, the nanoneedle was retracted from the cell. As the release is electrochemically controlled, we may program the release by adjusting the magnitude and duration of the applied electrical potential. An added benefit of this strategy is that the full electrochemical desorption of the SAM regenerates the gold surface for the reuse of the nanoneedle.

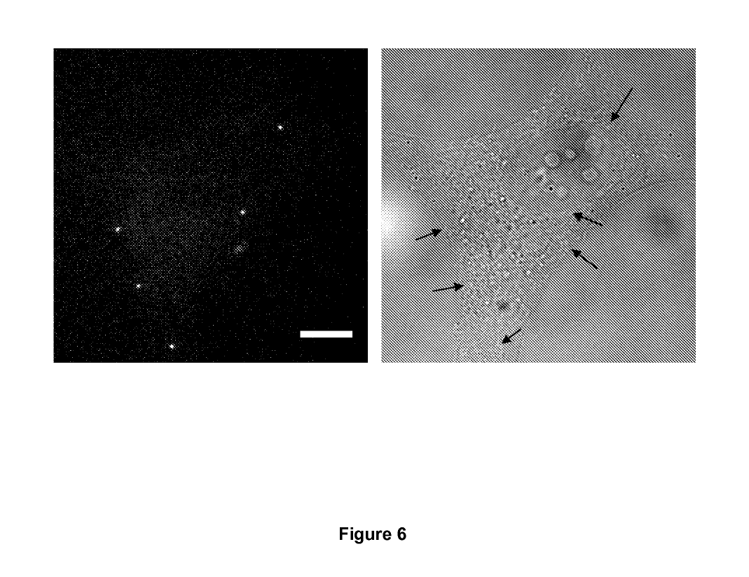

After the delivery, we imaged the target cell using fluorescence microscopy. We detected both slow-moving and fast-moving QDs within the boundary of the nucleus, showing their confinement in the nucleus (FIG. 5a). The size limit of the passive diffusion of materials through the nuclear pore complex also excludes the diffusive introduction of the QDs from the cytoplasm into the nucleus.⁽¹²⁾ Additionally, when we delivered QDs specifically into the cytoplasm, the QDs were confined in the cytoplasm (see FIG. 6).⁽¹⁷⁾ Because of the substantial reduction of the background signal from the out-of-focus QDs, as a result of delivering a tiny amount of QDs, we could detect single QDs even with a simple epifluorescence microscope. The release of QDs in the cell occurred only when an electrical potential above a certain magnitude was applied, confirming both the release of QDs through the electrochemical desorption of the SAM and the initial stable attachment of QDs on the nanoneedle.⁽¹⁷⁾ We did not observe any distinct difference between the tested cells and their neighboring cells during the time span of monitoring the cells (˜30 minutes); the tested cells remained viable after the penetration of the nanoneedle and the application of the electrical potential as assessed by the cell shape and the trypan blue assay. The charge introduced into the cell via the nanoneedle in the electrochemical release process did not perturb the physiological milieu of the cell

To determine whether this method can deliver singly-dispersed QDs, we measured the fluorescence intensity of slow-moving QDs. The detection of fast-moving QDs, especially those with low fluorescence, was still difficult due to their random diffusion in the three-dimensional environment of cell (this also made difficult the absolute quantification of the already tiny amount of delivered QDs). The acquired blinking behavior, typically only present for single QDs, indicated that the QDs were mostly single (FIG. 5b).^{(3, 6, 9, 17)} Small QDs clusters were also seen near the release site, mostly less mobile, resulted probably from their trapping to intracellular structures upon release. To demonstrate the capability of tracking single QDs and determine the dynamic behavior of the QDs in the nucleus, we applied the single-molecule tracking technique (FIG. 5c). The mean-square displacement (MSD) of moving QDs showed that QDs could freely diffuse in the nucleoplasm at a similar rate (with a diffusion coefficient D of ˜0.01-2 μm²/s, mean=0.5±0.7 μm²/s, n=8) as in the cytoplasm (D=˜0.1-4 μm²/s);⁽¹⁷⁾ but, these values might be underestimated as overall it was more difficult to track fast-diffusing QDs than slow-diffusing ones. The tracking of the diffusive QDs can also be used to probe the local physical properties within the nucleus by bio-microrheology (the nuclear delivery of probe particles has been a major obstacle in nuclear bio-microrheology measurement).⁽²⁴⁾ The viscosity of fluid can be related to the diffusion coefficient according to the Stokes-Einstein relation, D=kT/(6πηr), where k is the Boltzmann's constant, T is the absolute temperature, n is the viscosity, and r is the radius of the particle (QDs). Taking r to be ˜25.6 μm for QDs (estimated from the diffusion coefficient of QDs in aqueous solution of ˜17 μm²/s,⁽²⁵⁾, the “nanoscale” viscosity inside the nucleus was determined, from the locally measured diffusion coefficient, to be ˜5-2000 cP. These result suggests that the local viscosity in the region where QDs travel (probably, chromatin-poor domains) is similar to that of the cytoplasm that we measured previously,⁽¹⁷⁾ favorable for molecular transport processes through diffusion within the nucleus.^{(1, 26-28)} It also indicates that the physical environment of the nucleus is heterogeneous, consistent with published results.^{(1, 29)}

This spatially and now temporally controlled cellular delivery of QDs provides alternative strategies to study biology within the nucleus of living cells, which is otherwise technically challenging or not practical. For example, in combination with effective molecular targeting strategies of nanoparticles, which are already under active research,^{(4, 6, 10, 11)} (methods provided herein can potentially enable the use nanoparticles, such as quantum dots and magnetic nanoparticles, as molecular probes for simultaneous imaging and manipulation of single biomolecules (a major challenge in single-molecule studies)⁽³⁰⁾ in the nucleus and for observation of subnuclear structures and events.⁽²⁹⁾ In addition, this method offers a reliable means to deliver probe particles for bio-microrheology studies in cells and extend this methodology into the measurement of local physical properties within the nucleus (e.g., by using QDs as probe particles).⁽²⁴⁾ Technically, it is possible to apply this method for the direct intracellular delivery of biomolecules, such as proteins and DNA, which has been demonstrated previously outside cells.^{(21, 23)} (For these applications, although a recent study showed that proteins were still active after an electrical potential-induced release from a TiO₂ electrode (after applying a much higher electrical potential of 5 V than that used in our study),⁽³¹⁾ the more thorough examination of the effect of the applied electrical field on the functionality of the delivered biomolecules may be advisable, especially when the biomolecules are directly conjugated on the nanoneedle without a layer of insulating linker molecules. Finally, a major concern in the application of this method for cellular studies is its limited throughput. One way to overcome that is to automate the delivery process or adapt it to an array of nanoneedles^{(32, 33)}. For example, by integrating this electrochemical release concept with the delivery method demonstrated in a most recent study that showed the use of a substrate patterned with a large array of vertical and fixed Si nanowires for the efficient delivery of DNAs, RNAs, peptides, proteins and small molecules into cells⁽³³⁾, a spatially and temporally-programmed release and delivery into cells at a large scale may be realizable, if in such applications, the spatial precision (the sub-cellular precision) of the delivery is not of the chief requirement.

### Experimental Section

Surface Functionalization of Nanoneedles:

The gold-coated nanoneedle was prepared using a boron nitride nanotube as described previously.^{(17, 20)} A self-assembled monolayer (SAM) of biotin-terminated thiols was formed on the surface of the gold-coated nanoneedle by incubating the nanoneedle in 0.5 mM biotin-terminated tri(ethylene glycol)undecanethiol (Nanoscience instruments) or biotin-terminated tri(ethylene glycol)hexadecanethiol (Asemblon) in ethanol for 12 hours. Streptavidin-coated QDs (Qdot 655 streptavidin conjugates, Invitrogen) were conjugated by incubating the nanoneedle in 40 nM streptavidin-coated QDs in borate buffer (50 mM, pH 9.0) containing 1% bovine serum albumin for 30 minutes. A more detailed procedure is described previously.⁽¹⁷⁾

Cell Culture and Imaging:

HeLa cells were cultured in Dulbecco's modified Eagle's medium (DMEM) with 10% fetal bovine serum, 100 U/ml penicillin and 100 μg/ml streptomycin at 37° C. under 5% CO₂. Images were acquired using a Leica inverted epifluorescence microscope with a 63×1.32 numerical aperture oil-immersion objective, a charge-coupled device camera (C4742-95-12ERG, Hamamatsu), and a QD filter set for QD 655 (Chroma). The acquisition time for QD imaging was 50-500 ms. For the QD imaging inside the nucleus, to avoid the possibility of detecting QDs at the top or bottom of the nucleus, the nucleus envelop in the bright-field mode was identified and the cell on the same focal plane in the fluorescence mode was imaged.⁽¹⁷⁾

QD Tracking:

QDs were tracked in sequential fluorescence images of a cell using ImageJ.⁽³⁴⁾ The mean-square displacement (MSD) was computed as described previously.⁽¹⁷⁾

Cell Viability Tests:

Cell viability was examined using the trypan blue assay (0.4%, Invitrogen). After the penetration of the nanoneedle into cells and the application of a potential (1.4 V for 60 s), the cells were incubated at 37° C. under 5% CO₂ for about 10 minutes. After the incubation, individual cells were examined in serum-free medium with trypan blue (0.08%). The cells excluded the dye, indicating their viability (n=5). To track individual cells, cells were plated on gridded cell culture dishes for the viability tests.

Charge Injection During the Delivery Process

The number of charges injected into a cell by applying a potential (˜1.4 V for 60 s) is estimated to be ˜10⁷, assuming a current density of ˜10 μA/cm² (^{Ref. 35}) and an effective electrode surface area of ˜3×10⁻⁹ cm² (for a nanoneedle segment of ˜50 nm in diameter and ˜2 μm in length). The number of thiols on the nanoneedle segment is ˜10⁶, assuming the surface density of thiols of 7.7×10⁻¹⁰ mol/cm² (^{Ref. 36}). These numbers are only ˜0.01-0.001% of the typical number of ions in a cell (˜10¹¹); thus, their effect on the cellular environment is not believed to be substantial. Alternatively, to maintain the electrical neutrality of the intracellular environment, alternative pulses of positive and negative potentials can be applied during the release process. The strength of the electric field is ˜1 V/cm, much smaller than that used in electroporation (˜250-750 V/cm).⁽³⁷⁾

Factors Affecting the Success Rate of the Delivery

We detected QDs in about 60% of target cells after the delivery process (12 out of 21 cells). The failure in some cases is mostly due to the unreliable electric connection at the contact junction between the coated nanotube and the macroscopic metal wire (the electrochemically sharpened tungsten wire). As no electric potential can then be applied directly onto the nanoneedle, no electrochemical release of the quantum dots from such nanoneedles can be enabled. The fabrication process itself may introduce some unreliable electric connection, but it is believed the mechanical bending or even buckling experienced by the nanoneedle during the immersion of the nanoneedle into the cell medium that degrades the electric connection at the contact junction⁽³⁸⁻⁴¹⁾. The surface tension experienced by the nanoneedle during its entry to the cell medium is noticeable. Improving both the mechanical and electrical integrity of the contact junction between the nanoneedle and the macroscopic metal wire is expected to improve the success rate of the delivery. Another related cause affecting the success rate of the delivery is that not all QDs delivered into cells are fluorescent (the percentage can range from 30 to 70% according to literatures)^{(5, 6)}. Considering also that only a small number of QDs are delivered in each delivery and tracking fast-moving single QDs in a three-dimensional cellular environment is generally difficult, we may not fully count all successful deliveries.

Also see Yum et al., 2010, Small, 6, No. 19, 2109-2113, hereby incorporated by reference.

### REFERENCES

(1) T. Misteli, E. Soutoglou, Nat. Rev. Mol. Cell. Biol. 2009, 10, 243.
(2) F. Chen, D. Gerion, Nano Lett. 2004, 4, 1827.
(3) X. Michalet, et al., Science 2005, 307, 538.
(4) U. Resch-Genger, M. Grabolle, S. Cavaliere-Jaricot, R. Nitschke, T. Nann, Nat. Methods 2008, 5, 763.
(5) J. Delehanty, H. Mattoussi, I. Medintz, Anal. Bioanal. Chem. 2009, 393, 1091.
(6) M. Howarth, K. Takao, Y. Hayashi, A. Y. Ting, Proc. Natl. Acad. Sci. U.S.A. 2005, 102, 7583.
(7) M. Dahan, S. Levi, C. Luccardini, P. Rostaing, B. Riveau, A. Triller, Science 2003, 302, 442.
(8) S. Courty, C. Luccardini, Y. Bellaiche, G. Cappello, M. Dahan, Nano Lett. 2006, 6, 1491.
(9) H. Bannai, S. Levi, C. Schweizer, M. Dahan, A. Triller, Nat. Protocols 2007, 1, 2628.
(10) M. Howarth, W. Liu, S. Puthenveetil, Y. Zheng, L. F. Marshall, M. M. Schmidt, K. D. Wittrup, M. G. Bawendi, A. Y. Ting, Nat. Methods 2008, 5, 397.
(11) V. Roullier, S. Clarke, C. You, F. Pinaud, G. r. Gouzer, D. Schaible, V. r. Marchi-Artzner, J. Piehler, M. Dahan, Nano Lett. 2009, 9, 1228.
(12) A. M. Derfus, W. C. W. Chan, S, N. Bhatia, Adv. Mater. 2004, 16, 961.
(13) G. Ruan, A. Agrawal, A. I. Marcus, S, Nie, J. Am. Chem. Soc. 2007, 129, 14759.
(14) C. Tekle, B. van Deurs, K. Sandvig, T.-G. Iversen, Nano Lett. 2008, 8, 1858.
(15) D. J. Stephens, R. Pepperkok, Proc. Natl. Acad. Sci. U.S.A. 2001, 98, 4295.
(16) S. M. Rozenzhak, M. P. Kadakia, T. M. Caserta, T. R. Westbrook, M. o. Stone, R. R. Naik, Chem. Commun. 2005, 2217.
(17) K. Yum, S, Na, Y. Xiang, N. Wang, M.-F. Yu, Nano Lett. 2009, 9, 2193.
(18) X. Chen, A. K is, A. Zettl, C. R. Bertozzi, Proc. Natl. Acad. Sci. U.S.A. 2007, 104, 8218.
(19) S.-W. Han, C. Nakamura, N. Kotobuki, I. Obataya, H. Ohgushi, T. Nagamune, J. Miyake, Nanomedicine 2008, 4, 215.
(20) K. Yum, H. N. Cho, J. Hu, M.-F. Yu, ACS Nano 2007, 1, 440.
(21) P. Mali, N. Bhattacharjee, P. C. Searson, Nano Lett. 2006, 6, 1250.
(22) C. A. Canaria, J. So, J. R. Maloney, C. J. Yu, J. O, Smith, M. L. Roukes, S. E. Fraser, R. Lansford, Lab Chip 2006, 6, 289.
(23) S. Huang, E. Schopf, Y. Chen, Nano Lett. 2007, 7, 3116.
(24) D. Weihs, T. G. Mason, M. A. Teitell, Biophys. J. 2006, 91, 4296.
(25) D. Grünwald, A. Hoekstra, T. Dange, V. Buschmann, U. Kubitscheck, ChemPhysChem 2006, 7, 812.
(26) R. D. Phair, T. Misteli, Nature 2000, 404, 604.
(27) Y. Shav-Tal, X. Darzacq, S. M. Shenoy, D. Fusco, S. M. Janicki, D. L. Spector, R. H. Singer, Science 2004, 304, 1797.
(28) D. Y. Vargas, A. Raj, S. A. E. Marras, F. R. Kramer, S. Tyagi, Proc. Natl. Acad. Sci. U.S.A. 2005, 102, 17008.
(29) K. E. Handwerger, J. G. Gall, Trends Cell Biol. 2006, 16, 19.
(30) K. C. Neuman, A. Nagy, Nat. Methods 2008, 5, 491.
(31) Y.-Y. Song, P. Roy, I. Paramasivam, P. Schmuki, Angew. Chem. 2010, 122, 361; Angew. Chem., Int. Ed. Engl. 2010, 49, 351.
(32) S. Park, Y.-S. Kim, W. B. Kim, S. Jon, Nano Lett. 2009, 9, 1325.
(33) A. K. Shalek, et al., Proc. Natl. Acad. Sci. U.S.A., DOI: 10.1073/pnas.0909350107.
(34) I. F. Sbalzarini, P. Koumoutsakos, J. Struct. Biol. 2005, 151, 182.
(35) F. Loglio, M. Schweizer, D. M. Kolb, Langmuir 2003, 19, 830.
(36) P. Mali, N. Bhattacharjee, P. C. Searson, Nano Lett. 2006, 6, 1250.
(37) J. Sambrook, D. W. Russell, Molecular Cloning: A Laboratory Manual, 3 ed., Cold Spring Harbor Laboratory Press, Cold Spring Harbor, N.Y., 2001.
(38) I. U. Vakarelski, S. C. Brown, K. Higashitani and B. M. Moudgil, Langmuir, 2007, 23, 10893.
(39) J. E. D. de Asis, Y. Li, R. Ohta, A. Austin, J. Leung and C. V. Nguyen, Appl. Phys. Lett., 2008, 93, 023129.
(40) M. J. Esplandiu, V. G. Bittner, K. P. Giapis and C. P. Collier, Nano Lett., 2004, 4, 1873.
(41) Y. Narui, D. M. Ceres, J. Chen, K. P. Giapis and C. P. Collier, J. Phys. Chem. C, 2009, 113, 6815.

1. A method of controlled release of an agent into an intracellular environment of a biological cell, said method comprising the steps of:
a) providing a needle nanoelectrode;
b) attaching the agent to an outer surface of the needle nanoelectrode through a linking molecule, wherein the attachment comprises an electroactive chemical bond;
c) penetrating a cellular membrane with the needle nanoelectrode to position at least a portion of the nanoelectrode in the intracellular environment; and
d) applying an electric potential to the needle nanoelectrode to break the electroactive chemical bond, thereby controllably releasing the agent to the intracellular environment.

2. The method of claim 1, wherein at least a portion of the nanoelectrode surface is metallic, the linking molecule comprises a thiol end group and the electroactive bond is formed between the metallic portion of the electrode surface and the thiol group.

3. The method of claim 2, wherein at least a portion of the metallic nanoelectrode surface is functionalized by chemisorption of at least one type thiol molecule on the surface to form a self-assembled monolayer.

4. The method of claim 1, wherein the delivery portion of the nanoelectrode comprises a solid needle having a diameter that is from 50 nm to 300 nm.

5. The method of claim 4 wherein the needle comprises a nanotube coated with a gold layer, wherein the coated nanotube has a diameter that is from 50 nm to 100 nm.

6. The method of claim 4 wherein the solid needle comprises a metallic nanowire, a portion of the nanowire having an average diameter from 50 to 300 nm.

7. The method of claim 4, wherein the agent is attached to the nanoelectrode over an attachment region that is less than or equal to 10 μm from the delivery end of said needle.

8. The method of claim 4, wherein the agent is attached to the needle over an attachment contact area that is less than or equal to 3 μm².

9. The method of claim 1, further comprising removing the nanoelectrode needle from the intracellular environment.

10. The method of claim 9, wherein the agent comprises a detectable tag, said method further comprising:
e) detecting the detectable tag to monitor the distribution of the agent in the intracellular environment.

11. The method of claim 10, wherein the biological cell remains viable.

12. The method of claim 1, wherein the biological cell comprises an isolated cell.

13. The method of claim 1, wherein the controlled release is in a biological cell that is in vitro, in vivo or ex vivo.

14. The method of claim 1, wherein at least a portion of the nanoelectrode surface is metallic and the attachment step further comprises functionalizing an exposed portion of the metallic nanoelectrode surface by:
i) forming a self-assembled monolayer comprising a first and a second thiol species on the exposed portion of the nanoelectrode surface by introducing to the metallic nanoelectrode surface the first and the second thiol species, wherein the first thiol species comprises a biotin end group and the second thiol component does not comprise a biotin end group;
ii) attaching a biotin-binding protein functionalized agent moiety to at least a portion of the first thiol species in the monolayer by contacting the self-assembled monolayer with the biotin-binding protein functionalized agent moiety, thereby attaching the agent moiety to the surface of the nanoelectrode.

15. The method of claim 14, wherein the molar percentage of the first thiol species, relative to the second thiol species, is less than 100% and greater than or equal to 5%.

16. The method of claim 14, wherein the concentration of agent moieties in the functionalized region is from 1.0×10⁻¹⁰ mol to 10.0×10⁻⁹ mol per 1 cm².

17. The method of claim 14, wherein the functionalized nanoelectrode surface has geometry that is cylindrical.

18. The method of claim 14, wherein the self-assembled monolayer is formed by dipping the nanoelectrode in a solution that contains a mixture of the first and second thiol species.

19. The method of claim 18, wherein the dipped portion of the nanoelectrode comprises a needle having a diameter that is less than or equal to 100 nm.

20. (canceled)

21. The method of claim 1 wherein the penetrating step is by a micromanipulator operably connected to the nanoelectrode.

22. The method of claim 1 wherein the agent comprises a biological molecule.

23. The method of claim 1, wherein the agent is released in a time-release period that is less than or equal to 90 seconds.

24. The method of claim 1 wherein the absolute value of the electric potential used to electrochemically break the thiol bond is less than or equal to 1.5V versus an Ag/AgCl standard reference electrode.

25. The method of claim 1 wherein the nanoelectrode is reused.

26. The method of claim 1, wherein the intracellular environment is the nucleus or the cytoplasm.

27. The method of claim 1 wherein the intracellular environment is sub-nuclear.
